(19) United States
(12) Patent Application Publication
(10) Pub. No.: US 2003/0097107 A1
(43) Pub. Date: May 22, 2003

(54) Absorbent article with increased convective gas flow rates therethrough

(75) Inventors: Cornelia Sprengard-Eichel, Frankfurt (DE); Matthias Konrad Hippe, Sulzbach (DE); Bruno Johannes Ehrnsperger, Bad Soden (DE); Mattias Schmidt, Idstein (DE)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/325,235

(22) Filed: Dec. 19, 2002

Related U.S. Application Data
(63) Continuation of application No. PCT/US00/17084, filed on Jun. 21, 2000.

(51) Int. Cl. A61F 13/15; A61F 13/20

(52) U.S. Cl. 604/378; 604/367

(57) ABSTRACT

The present invention relates to absorbent articles providing improved aeration between the article and the wearer's skin during use. The absorbent articles allow for convective gas/air transport therethrough by providing absorbent cores having sufficient basis capacity simultaneous with air/gas permeability.

This is a continuation of International Application PCT/US00/17084, with an international filing date of Jun. 21, 2000.

The present invention relates to disposable absorbent articles, such as baby diapers, adult incontinence articles, and in particular to such articles providing improved aeration during use.

### BACKGROUND

Disposable, absorbent articles such as diapers, incontinence articles, sanitary towels, training pants and the like are well known in the art. Typically, disposable absorbent articles comprise a liquid pervious topsheet that faces the wearer's body, a liquid impervious backsheet that faces the wearer's clothing, an absorbent core interposed between the liquid previous topsheet and the backsheet, and means to keep the core in fixed relation to the wearer's body.

Numerous attempts have been disclosed aiming at improving on the skin condition of the wearer by allowing the over-hydrated skin to dehydrate to an acceptable level by allowing either air to reach the skin thus minimizing potential occlusion effects, and/or by water vapor being removed from the surface of the skin. Generally, such mechanisms are referred to as “breathability” or “vapor or moisture permeability”.

A number of such applications aim at feminine hygiene products, such as catamenial products or so-called “panty-liner” as described in EP-A-0.104.906; EP-A-0.171.041; EP-A-0.710.471. WO97/23182 further discloses an absorbent structure comprising fibrous superabsorbent material and combining such breathable materials with fibrous superabsorbent material in the absorbent core. Such products generally have relatively low fluid storage capacity when compared to baby diapers or adult incontinence products, often being designed for theoretical capacities significantly exceeding the ones for the feminine hygiene products.

Breathable materials include various kinds of webs, such as films, which were rendered air and/or vapor permeable by aperturing as described in U.S. Pat. No. 5,628,737, or by exploiting the “microporosity” property as described in EP-A-0.238.200; EP-A-0.288.021; EP-A-0.352.802; EP-A-0.515.501; U.S. Pat. No. 4,713,068, whereby small voids are created within the film similar to very small cracks. WO 94/23107; WO 94/28224; U.S. Pat. No. 4,758,239; EP-A-0.315.013 all describe alternative breathable materials which include fibrous textile or non-woven webs with air or vapor easily penetrating through the relatively large pores of the structure. Such webs can be either untreated or treated with regard to improving their liquid impermeability properties as described in EP-A-0.196.654. In WO 95/16562 a laminate of a non-woven with a breathable film is disclosed. Further disclosures such as in WO 95/16746 relate to other materials allowing water molecules to diffuse therethrough. Also, combinations of various materials comprising various layers of any of the above elements are also well known.

Also, for articles designed for receiving higher amounts of liquids, such as baby or adult incontinence diapers, other approaches are available aimed at keeping only part of the article breathable, such as by covering the liquid absorbing parts (often referred to as absorbent core) by a non-breathable material, but having other parts of the article made of breathable materials, see e.g. EP-A-0.059.503 (Obenour).

There have been many attempts to improve the fluid handling properties of absorbent articles or cores, particularly where requirements such as a reduction of product bulkiness or thickness is desired. Such effects are discussed in European Patent Application 96105023.4 filed on Mar. 29, 1996, but also in U.S. Pat. No. 4,898,642; EP-A-0.640.330; EP-A-0.397.110; EP-A-0.312.118.

PCT publication WO 98/58609 discloses a disposable absorbent article sustaining low vapor phase moisture in the space enclosed between the article and the wearer in use. Such can be evaluated by measuring relative humidity on a laboratory mannequin, by combining high performance; low rewet absorbent cores with very breathable backsheet materials. Thus, the thrust of this disclosure aims at providing an absorbent article with good liquid retention in the cores, combined with water vapor permeable, liquid impermeable barrier materials for the use as backsheets. The preferred, specific embodiment of this disclosure is directed towards the use of a high amount of absorbent capacity so as to dry out the structures close to the skin of a wearer.

A series of related and co-filed PCT applications (WO 00/10497; WO 00/10498, WO 00/104099, WO 00/10500, WO 00/10501) relates to breathable absorbent articles, including when these are in the wet state. One approach described therein relates to creation of high permeability zones within an absorbent core, such as by aperturing the absorbent core, or by creating portions in the core containing substantially less high absorbency material than other portions of the core. Overall, the gas transfer mechanisms rely on gas diffusion mechanism, such as demonstrated by the preferred use of microporous film materials, as well as by the Tracer Gas Test. The approaches described therein can provide relatively good relative humidity conditions while being worn, as long as the article is not loaded with urine. However, upon loading these approaches exhibit significantly increased relative humidity conditions.

Thus, the prior art failed to provide satisfactory solutions for absorbent cores where the the ultimate storage capacity (the capacity required to absorb the expected loading during the intended use) is exceeded by a minimal amount. The ultimate storage capacity should preferably not be more than about twice the design capacity of the article.

The prior art also failed to provide structures which provide good convective transport without unduly complicating the manufacturing process, which is the case for strongly inhomogeneous structures such as absorbent cores with apertures or ventilation openings.

Consequently, there is a need for absorbent articles, wherein the micro climate and especially the relative humidity is kept within ranges as generally accepted as being comfortable, namely between 30% to 50%. In addition, there is a need to provide articles, wherein the relative humidity is kept within this range even upon wetting of the article. Further, there is a need to achieve such goals without unduly complicating the structure, i.e. by avoiding designs using high amounts of absorbent, and/or by creating strongly inhomogeneous structures, such as cores comprising apertures. Particularly, there is a need for absorbent articles, wherein good microclimate conditions are achieved by carefully designing the chassis elements.

### SUMMARY

The present invention provides an absorbent article with improved performance by providing articles with good “Wet Article—Relative Humidity differential” as being descriptive for the climate differences between the environment and the space between the article and the wearer. Preferably, the article comprises a backsheet which is air or gas permeable, but under normal use conditions is not liquid permeable. The absorbent core can have an ultimate liquid storage capacity, which is preferably not excessive when compared to the design capacity of the article, though it preferably exhibits a basis capacity of more than about 0.7 ml/cm². The core should further allow convective gas or air transport therethrough, exhibiting a permeance of at least 0.1 Darcy/mm, preferably of more than 1.0 Darcy/mm, particularly when being loaded and wetted. The article according to the present invention is particularly suitable for use as hygienic disposable absorbent articles, such as baby diapers, and adult incontinence garments, in order to provide a comfortable microclimate in the space between the article and the wearer.

In a particular design, the absorbent core can comprise a liquid storage region and a liquid acquisition/distribution region positioned between this liquid storage region and the topsheet, whereby this acquisition/distribution region comprises an evaporation barrier layer/region, so as to reduce the evaporation tendency of the article from the core towards the space between the article and the wearer during the intended use. The acquisition/distribution region may contain material having a drip capacity of at least 5 g/g, which can comprise cellulosic fibrous material.

The article may include a bellows, which is repeatedly deformable to force airflow through the absorbent article in a controlled manner.

### SHORT DESCRIPTION OF THE DRAWINGS

FIG. 1—schematic diagram of the Dynamic Impact test method,

FIG. 2—schematic diagram of the Water Vapor Transmission method equipment,

FIG. 3—schematic drawing of the harness,

FIG. 4—harness on a mannequin,

FIG. 5—picture of the sensor box equipped with relative humidity sensor,

FIG. 6—acquisition test set up,

FIG. 7—post acquisition collagen rewet test set up,

### DETAILED DESCRIPTION

General Definitions

As used herein, the term “absorbent articles” refers to devices, which absorb and contain body exudates, and, more specifically, refers to devices which are placed against or in proximity to the body of the wearer to absorb and contain the various exudates discharged from the body. As used herein, the term “body fluids” includes, but is not limited to, urine, menses and vaginal discharges, sweat and feces.

The term “disposable” is used herein to describe absorbent articles which are not intended to be laundered or otherwise restored or reused as an absorbent article (i.e., they are intended to be discarded after use and, preferably, to be recycled, composted or otherwise disposed of in an environmentally compatible manner).

As used herein, the term “Z-dimension” refers to the dimension orthogonal to the length and width of the member, core or article. The Z-dimension usually corresponds to the thickness of the member, core or article. As used herein, the term “X-Y dimension” refers to the plane orthogonal to the thickness of the member, core or article. The X-Y dimension usually corresponds to the length and width, respectively, of the member, core or article.

As used herein, the term “absorbent core” refers to the component of the absorbent article that is primarily responsible for fluid handling properties of the article, including acquiring, transporting, distributing and storing body fluids. As such, the absorbent core typically does not include the topsheet or backsheet of the absorbent article.

As used herein, the term “absorbent member” refers to the components of the absorbent core that typically provide one or more fluid handling functionality, e.g., fluid acquisition, fluid distribution, fluid transportation, fluid storage. The absorbent member can constitute the entire absorbent core or only a portion of the absorbent core, i.e., the absorbent core can comprise one or more absorbent members. The “storage absorbent member” is the absorbent member component(s) of the absorbent core that function primarily to ultimately store absorbed fluids. As discussed above, the storage absorbent member may also distribute fluid as a result of its vertical wicking capability.

As use herein, the term “layer” refers to an absorbent member whose primary dimension is X-Y, i.e., along its length and width. It should be understood that the term layer is not necessarily limited to single layers or sheets of material. Thus the layer can comprise laminates or combinations of several sheets or webs of the requisite type of materials. Accordingly, the term “layer” includes the terms “layers” and “layered”. For purposes of this invention, it should also be understood that the term “upper” refers to absorbent members, such as layers, that are nearest to the wearer of the absorbent article during use, and typically face the topsheet of an absorbent article; conversely, the term “lower” refers to absorbent members that are furthermost away from the wearer of the absorbent article and typically face the backsheet.

All percentages, ratios and proportions used herein are calculated by weight unless otherwise specified.

All patent and publications referenced herein are hereby incorporated by reference.

Design Capacity

In order to be able to compare absorbent articles for varying end use conditions, or differently sized articles, the “design capacity” has been found to be a suitable measure. For example, babies are representing a typical usage group, but even within this group the amount of urine loading, frequency of loading, composition of the urine will vary widely from smaller babies (new-born babies) to toddlers on one side, but also for example among various individual toddlers. Another user group may be larger children, still suffering from a certain form of incontinence. Also, incontinent adults can use such articles, again with a wide range of loading conditions, generally referred to as light incontinence ranging up to severe incontinence. Henceforth, such articles capable of coping with such requirements should have the capability of picking up such amounts of urine, which will be referred to for the further discussion as “design capacity”.

These amounts of fluids have to be absorbed by materials, which can ultimately store the bodily fluids, or at least the aqueous parts of these, such that little fluid, if any, is left on the surface of the article towards the wearers skin. The term “ultimate” refers in one respect to the situation as in the absorbent article at long wearing times, in the other respect to absorbent materials, which reach their “ultimate” capacity when being equilibrated with their environment. This can be in such an absorbent article under real in-use conditions after long wearing times, or this also can be in a test procedure for pure materials or material composites. If the processes under consideration have asymptotic kinetic behavior, one skilled in the art will readily consider “ultimate” capacities to be reached when the actual capacity has reached a value sufficiently close to the asymptotic endpoint, e.g. relative to the equipment measurement accuracy.

An absorbent article can comprise materials which are primarily designed to ultimately store fluids, and other materials which are primarily designed to fulfill other functions such as acquisition and/or distribution of the fluid, but may still have a certain ultimate storage capability. Suitable core materials according to the present invention are described without attempting to artificially separate such functions. Nonetheless, the ultimate storage capacity can be determined for the total absorbent core, for regions thereof, for absorbent structures, or even sub-structures, and also for materials as being used in any of the previous.

In case of applying the present invention to other articles requiring different end-uses, one skilled in the art will be able to readily adopt the appropriate design capacities for other intended user groups. In order to determine or evaluate the Ultimate Design Storage Capacity of an absorbent article, a number of methods have been proposed. In the context of the present invention, it is assumed, that the Ultimate Storage Capacity of an article is the sum of the ultimate absorbent capacities of the individual elements or material. For these individual components, various well-established techniques can be applied as long as these are applied consistently throughout the comparison. For example, the Tea Bag Centrifuge Capacity as developed and well established for superabsorbent polymers can be used for such materials, but also for others. Once the capacities for the individual materials are known, the total article capacity can be calculated by multiplying these values (in ml/g) with the weight of the material used in the article. For materials having a dedicated functionality other than ultimate storage of fluids—such as acquisition layers and the like—the ultimate storage capacity can be neglected, either as such materials do in fact have only very low capacity values compared to the dedicated ultimate fluid storage materials, or as such materials are intended to not be loaded with fluid, and thus should release their fluid to the other ultimate storage materials.

With such definitions, for example a so-called “panty liner” product exhibits very low Ultimate storage capacities of a few ml or less. Feminine Hygiene pads have often up to about 20 ml, light urinary incontinence articles have for example 75 ml or about 90 ml , medium urinary incontinence articles, or also smaller baby diaper can have about 165 ml, and toddler size baby diapers reaching 300 ml or more, and severe adult incontinence article having 600 ml or more of ultimate storage capacity.

Teabag Centrifuge Capacity Test (TCC test)

While the Teabag Centrifuge Capacity (TCC) test has been developed specifically for superabsorbent materials, it can readily be applied to other absorbent materials. The Teabag Centrifuge Capacity test measures the Teabag Centrifuge Capacity values, which are a measure of the retention of liquids in the absorbent materials.

The absorbent material is placed within a “teabag”, immersed in a 0.9% by weight sodium chloride solution for 20 minutes, and then centrifuged for 3 minutes. The ratio of the retained liquid weight to the initial weight of the dry material is the absorptive capacity of the absorbent material. Two liters of 0.9% by weight sodium chloride in distilled water is poured into a tray having dimensions 24 cm×30 cm×5 cm. The liquid filling height should be about 3 cm. The teabag pouch has dimensions 6.5 cm×6.5 cm and is available from Teekanne in Düsseldorf, Germany. The pouch is heat sealable with a standard kitchen plastic bag sealing device (e.g. VACUPACK2 PLUS from Krups, Germany).

The teabag is opened by carefully cutting it partially, and is then weighed. About 0.200 g of the sample of the absorbent material, accurately weighed to +/−0.005 g, is placed in the teabag. The teabag is then closed with a heat sealer. This is called the sample teabag. An empty teabag is sealed and used as a blank.

The sample teabag and the blank teabag are then laid on the surface of the saline solution, and submerged for about 5 seconds using a spatula to allow complete wetting (the teabags will float on the surface of the saline solution but are then completely wetted). The timer is started immediately.

After 20 minutes soaking time the sample teabag and the blank teabag are removed from the saline solution, and placed in a Bauknecht WS130, Bosch 772 NZK096 or equivalent centrifuge (230 mm diameter), so that each bag sticks to the outer wall of the centrifuge basket. The centrifuge lid is closed, the centrifuge is started, and the speed increased quickly to 1,400 rpm. Once the centrifuge has been stabilized at 1,400 rpm the timer is started. After 3 minutes, the centrifuge is stopped.

The sample teabag and the blank teabag are removed and weighed separately.

The Teabag Centrifuge Capacity (TCC) for the sample of absorbent material is calculated as follows:

TCC=[(sample teabag weight after centrifuging)−(blank teabag weight after centrifuging)−(dry absorbent material weight)]÷(dry absorbent material weight).

Also, specific parts of the structures or the total absorbent articles can be measured, such as “sectional” cut outs, i.e. looking at parts of the structure or the total article, whereby the cutting is done across the full width of the article at determined points of the longitudinal axis of the article. In particular, the definition of the “crotch region” as described above allows to determine the “crotch region capacity” . Other cut-outs can be used to determine a “basis capacity” (i.e. the amount of capacity contained in a unit area of the specific region of the article. The size of the unit area (preferably 2 cm by 2 cm) the defines how much averaging is taking place—naturally, the smaller the size, the less averaging will occur.

Ultimate Storage Capacity

In order to determine or evaluate the Ultimate Design Storage Capacity of an absorbent article, a number of methods have been proposed. In the context of the present invention, it is assumed, that the Ultimate Storage Capacity of an article is the sum of the ultimate absorbent capacities of the individual elements or material. For these individual components, various well-established techniques can be applied as long as these are applied consistently throughout the comparison. For example, the Tea Bag Centrifuge Capacity as developed and well established for superabsorbent polymers (SAP) can be used for such SAP materials, but also for others.

Once the capacities for the individual materials are known, the total article capacity can be calculated by multiplying these values (in ml/g) with the weight of the material used in the article. For materials having a dedicated functionality other than ultimate storage of fluids—such as acquisition layers and the like—the ultimate storage capacity can be neglected, either as such materials do in fact have only very low capacity values compared to the dedicated ultimate fluid storage materials, or as such materials are intended to not be loaded with fluid, and thus should release their fluid to the other ultimate storage materials.

Basis Capacities

Each of the described capacities can also be expressed as a basis capacity, which is defined as the respective capacity per a unit area, expressed such as in ml/cm² or equivalents. This capacity can further be a local basis capacity, or an average over a certain area.

Microclimate

The term microclimate as used herein refers to the conditions of the space between the article and the wearer. In this context, this space is confined by the body of the wearer, generally the skin of the wearer, and the hygienic article, comprising the core region of the article, and the chassis regions, the latter generally being the peripheral regions. Frequently, the article comprises elastication elements, such as leg cuffs, or barrier cuffs. Such sealing elements can, but do not need to, reduce the liquid and air exchange between the outside or environment and the space between the article and the skin of the wearer.

Often, this space is a unitary, connected space, but it also can consist of sub-spaces, which can be connected to each other or which can be several spaces, which preferably all are designed and constructed according to the present invention, as applicable. The elements or the materials of the absorbent article are not considered to be part of this space, though such elements or materials may extend into such a space. Similarly, body elements are not considered to be included in the space. Also, liquid and/or solid body exudates, such as urine or feces, are not considered as part of this space. Consequently, the space can be described by the conditions of the gas. These conditions can have actual, local values (i.e. at one point in time at one location), or can be averaged over time or space or both.

The first element of the conditions for the gas space is the composition, and in particular, the water content expressed as relative humidity as defined by the ratio of the actual water vapor partial pressure to the corresponding water vapor partial pressure at saturation. However, other components such as odorous vapors, or skin attacking components can be contained in the space. The temperature in the space is also of importance, as it is impacting on the relative humidity, but also because of its impact on the skin condition, and comfort of the wearer. The temperature can be, but often will not be constant throughout the space. If the temperature of the skin of the wearer and the environment are not constant, there will be a temperature gradient across the article, across the space and versus the surface of the skin of the wearer. Typically, the temperature within the space will be between 30° C. and 36° C., and temperatures of about 34° C. are often perceived as comfortable.

It has been found, that in order to maintain comfortable and healthy skin, the microclimate within the space between the article and the wearer should be kept in the comfortable relative humidity range, preferably of less than 50% RH, more preferably of less than 45% RH and even more preferably less than 40% RH. However, in order to prevent underhydration of the skin of the wearer, the microclimate should not have less than about 20% RH, preferably not less than about 30%.

In order to achieve such preferred microclimate conditions, it has been found, that the article should—when submitted to the In-Vivo microclimate testing—exhibit a Wet Article Relative Humidity Differential of less than 20.0%, preferably less than 15.0%, and even more preferably less than 10.0%, as defined hereinafter.

A further important aspect of the gas space is the flow of the gas therein, in particular the convective transport in the gas phase. This flow is connected with local pressure changes in the space—while there will generally be no major pressure differential between the space and the environment (i.e. the region outside of the article when worn), already small changes in pressure, such as can be created by movements of the wearer, or a temperature and/or composition differential can cause convective flow such as through gaps between the article, and the wearer. Preferably, it also can take place through the article itself, such as through materials of the article. For articles according to the present invention, this convection can occur through the article along the z-direction of the article, though it may also include x-y directional components.

The convective transport can be measured and expressed by the flow speed or velocity (in m/sec), or by the flow rate (in g/sec), or by the area specific flux (in g/sec/cm2). Convective transport should be distinguished from diffusive transport. The latter generally has much lower transport rates, and can—for example—be achieved by moisture transport through a barrier layer, such as by using so-called monolithic films as can be made from materials as Hytrel™ , as available from DuPont, or by slow migration of vapor through a microporous film material. Known elements for allowing convective transport through certain elements of an article are very open materials in the non-absorbent (chassis) parts of the article, such as nets, or scrims, or non-wovens with a sufficiently high permeability and permeance for gas (as discussed hereinafter). Such transport mechanism is also known to go through an absorbent structure, such as when aperturing the core as described in the above mentioned series of PCT publications (WO 00/10497; WO 00/10498, WO 00/104099, WO 00/10500, WO 00/10501), whereby the general teaching of these documents does not direct to convective transport through the article, but on diffusive overall transport—such as implied by the preferred use of microporous backsheets.

In one aspect, the present invention aims at providing z-directional convective transport through the complete absorbent article in the region of where the liquid is absorbed, i.e. through the absorbent core, even when this is loaded, and in particular not through special “venting means” as disclosed in the prior art but rather through the absorbent material itself. Henceforth, in addition to highly permeable backsheet and topsheet materials, the article requires an absorbent core, which has a sufficiently high permeance in the absorbent material even when being loaded.

In another aspect, the present invention relates to particularly enhanced convection through gaps or the article such as described in more detail in U.S. Pat. No. 6,450,997B1 issued to Seitz, et al. titled “ABSORBENT ARTICLE HAVING A BELLOWS FOR CIRCULATING FRESH AIR”, disclosing enhanced circulation by means of bellows.

The particularly useful absorbent structures for the present invention combine both functionality of the liquid absorbency with the convective gas transport through this structure and the remaining elements forming the absorbent article at the same time.

Thus, the ability for convective flow through the structure should not be created by inhomogeneities in the structure such as by providing apertures, or particular regions with enhanced ability for convective flow at reduced capacity.

The convective transport through dry and or loaded articles can be assessed by the Gas Permeability methods, as described herein, whereby permeability values for dry and wet articles can be determined. In combination with respective caliper measurements (on the dry and/or wet article, respectively), the permeance of the structure can be calculated, by dividing the permeability by the thickness of the structure. For inhomogeneous structures, the sample preparation or the test setup might require adaptation so as to not measure through the “venting channels” such as apertures and/or low basis weight, and/or low basis capacity regions. Preferably, an article according to the present invention provides for wet permeance of more than about 0.1 Darcy/mm, preferably more than about 0.5 Darcy/mm, and even more preferably more than about 1.0 Darcy/mm. Typically, the respective dry article permeance is less than the permeance of the wetted article.

As the core of the article will typically provide an important resistance to the convective flow through the article, the core should exhibit a sufficiently high wet permeance of more than about 0.1 Darcy/mm, preferably more than about 0.5 Darcy/mm, and even more preferably more than about 1.0 Darcy/mm. The respective dry core permeance should not be less than the wet one, and is typically more than 1 Darcy/mm or even more than 10 Darcy/mm.

Suitable core structures for such articles can be formed according to many known ways, and incorporate many known materials, such as comprising fibrous materials, such as cellulose or synthetic fibers, or particulate materials, such as superabsorbent particles, or foams, and especially foams formed by the High Internal Phase Emulsion polymerization process, or combinations thereof. The combinations can be homogeneous mixtures thereof, or segregated or separated materials. The openness of such structures can be achieved by selecting particular arrangements of permeable materials. It has been found, that superabsorbent materials are particularly suited to be used in articles according to the present invention, if they exhibit high Saline Flow Conductivity performance (SFC), preferably of more than 30*10⁷ cm³ sec/g, when evaluated according to the disclosure of U.S. Pat. No. 5,599,335, which is incorporated herein by reference. Such materials can be arranged in a homogeneous mixing with fluff pulp, or can be layered between suitably open and permeable layers of porous materials, such as tissues, especially if these are air-laid, or nonwoven materials. Particularly suitable materials are superabsorbent materials as described in the above referenced U.S. Pat. No. 5,599,335, when arranged in a homogeneous blend with conventional fluff pulp, at a concentration of 50% superabsorbent, preferably 80% and even more preferably more than 90% concentration based on the weight of the superabsorbent/fluff mixture. Suitable mixtures can further exhibit densities of between 0.1 g/ cm³ and 0.3 cm³, preferably between 0.15 cm³ and 0.2 cm³.

In particular embodiments, such mixtures can comprise means, which enhance the integrity of the mixture, especially in the dry state. Thus, low amounts of adhesive may be added to the mixture, or other binders, such a thermobondable synthetic fibers.

In addition to the liquid storage elements in the core, the core may comprise other liquid-handling members, such as for enhancing fluid acquisition, or distribution.

Suitable cores are further described in EP-A-0.774.242; PCT applications IB99/00739, IB99/00741, IB99/00751, all filed on Apr. 23, 1999; PCT Application US98//05044, filed on Mar. 18, 1998. The storage core may further comprise polymeric porous materials, preferably made by the High Internal Phase Emulsion Polymerization process (“HIPE” foams), such as described in PCT applications IB99/00404 and IB99/00408, both filed Mar. 12, 1999, all these publications are incorporated herein by reference. Optionally, and often preferred, the storage core can be enveloped by a suitable web, such as a paper tissue or a suitable non-woven material, such as described in WO 97/07761 and in PCT application IB99/00689, filed on Apr. 16, 1999, which is incorporated herein by reference.

A further suitable core structure comprises an acquisition/distribution member which includes an evaporation barrier, such as an apertured formed film, as described in more detail in co-filed PCT application “Disposable absorbent articles having low rewet and reduced evaporation from the core through the topsheet”, attorney docket number CM2375.

The absorbent core may further comprise elements, which are particularly designed to handle non-urinary excretions, for example feces. At least as long as such elements are only loaded with liquid excretions, such as urine, these preferably satisfy the permeance requirements as described in the above.

This permeance of the absorbent core is preferably achieved in the regions of the article providing absorbent capacity. While it is preferred for material usage efficiency not to have articles with an excessive overall capacity, the basis capacity (i.e. the amount of ultimate liquid storage capacity per unit area) should not be less than 0.3 ml/cm², preferably not be less than 0.6 ml/cm². This basis capacity and the corresponding permeance can be readily determined for structures, where sufficiently homogeneous regions are sufficiently large in dimension and size so as to allow testing. In situations, where these areas are too small to allow direct measurement thereof, the material may be modified so as to allow assessment thereof. For example, apertures may be blocked (i.e. filled with inert material, or structures may be rearranged close large apertures, (obviously with careful monitoring of the density and the caliper).

Other Article Elements

In addition to the described absorbent core, the absorbent article comprises a backsheet to separate the core from the outside of the article. The term backsheet refers to any material, or layer, or coating, positioned between the core and the environment in the direction away from the wearer. Functionally, the backsheet must on one side satisfy the functional requirement of retaining the liquid as deposited onto and into the article, as well as being capable of allowing gas or vapor flow rates therethrough which should preferably be not the rate-limiting step of gas transfer from the inner space to the outside. In addition, the backsheet may satisfy further functions, such as providing stability and integrity to the article, or providing a pleasant feel or hand, or masking of exudates.

Preferably, the backsheet has a WVTR of at least 3000 g/24 hrs/m², preferably of more than 2800 g/24 hrs/m², and even more preferably of more than 4000 g/24 hrs/m² when submitted to the WVTR test as described hereinafter.

The backsheet should further prevent liquids from soiling the outside therethrough and hence, are designed to a leakthrough value of less than 100 g/m², preferably less than 50 g/m², and even more preferably of less than 10 g/m², when submitted to the Dynamic liquid impact test, and a polyhole rewet performance of less than 0.10 mg, preferably less than 0.05 mg, and even more preferably less than 0.01 mg, when submitted to the polyhole rewet test, as described hereinafter.

The backsheet material can be a single layer made of homogeneously or inhomogeneously distributed phases, or a two- or multilayer construction. The backsheet material can be a porous material, such as a film with a plurality of apertures, or it can be a porous web such as a non-woven or a foam material. The backsheet thus maybe constructed from a variety of materials and or composites. For example, the backsheet may be made of polymeric film materials, suitably apertured to provide the required breathability without jeopardizing the leak-through performance. The backsheet may be made of non-woven materials, or multi-layer nonwovens, for instance, well-known barrier webs, such as composites comprising a spun-bonded layer and a meltblown layer. Suitable materials include three-dimensionally formed apertured films, preferably comprising slanted cones, as described in PCT applications US99/02395or US99/02393, both filed on Feb. 3, 1999 and both incorporated herein by reference. Such films may be combined with non-wovens to form laminates. The backsheets or components thereof may be attached to each other or to other elements of the article. For example, when the backsheet is a composite made of an apertured film material with a non-woven web, the film material may be attached to the core components. The non-woven can also be attached to the film over the full area of the backsheet, but preferably the layers are only attached to each other in the peripheral regions of the article.

Further, the backsheet can be a porous material comprising swellable substances, such as superabsorbent materials and the like, as described in PCT publication WO 97/23182. In yet a further embodiment, the backsheet material or at least parts thereof are rendered hydrophobic, such as by applying fluourocarbon treatments as described in PCT publication WO 00/14229 (Palumbo).

Exemplary backsheet materials are as follows:

Sample BS-1 is a nonwoven composite made of melt-blown and spunbonded layers as provided by BBA-COROVIN, Peine, Germany, under the designation MD3000, and exhibits at a basis weight of about 12 gsm a WVTR of about 4670 [g/m²/24 hr]. When testing a double layer of this material, the WVTR value is about 4470 [g/m²/24 hr]. When submitted to a permeability test by using the “Textiluhr nach Kretschmar”, Sample BS-1 provided a permeance result of about 375 Darcy/mm.

Sample BS-2 is an apertured formed film with slanted cones, available from Tredegar under the designation V174 LD40, exhibiting a WVTR value of about 2850 [g/m²/24 hr].

Sample BS-3 is a combination of a layer of sample BS-1 with the film of Sample BS-2, providing a WVTR of about 2850 [g/m²/24 hr], demonstrating, that the formed film resistance to flow dominates. When submitting sample BS-2 to the “Textiluhr nach Kretschmar” testing, it provided a result of about 87 Darcy/mm.

Further backsheet samples have been submitted to the permeability and caliper testing to determine their permeance. As none of the methods used was able to provide useful results over the full range of permeabilities, different methods have been selected to provide the data, however, the resulting permeance values as expressed in Darcy/mm are comparable across the whole range of selected materials.

Sample BS-4 (RR-1) is a typical microporous films, e.g. as available from FinoTech under the designation BSB-X3-330, then mechanically activated to provide a MVTR value of about 1500 [g/m²/24 hr]. When using the PMI permeameter, the permeance was determined to be 0.0003 Darcy/mm.

Sample BS-5 (RR-2) is a further typical microporous film of the same type, but mechanically activated to then provide a MVTR value of about 3500 [g/m²/24 hr]. When using the PMI permeameter, the permeance was determined to be 0.0005 Darcy/mm.

Sample BS-6 (MDO) as available from Tredegar Inc. under the designation X25498 or X25620 and exhibiting a MVTR of about 3500 [g/m²/24 hr] was evaluated according to the PMI method, and gave a permeance of 0.0024 Darcy/mm.

Sample BS-7 (CDO) as available from EXXON under the designation EXXAIRE and exhibiting a MVTR of about 3800 [g/m²/24 hr] was evaluated according to the PMI method, and gave a permeance of 0.0029 Darcy/mm.

Sample BS-8, being an alternative composite PP-non-woven as available from BBA-COROVIN, Peine, Germany, under the designation MD2005, 5BSSB gave upon testing according to the “Textiluhr nach Kretschmar” test a permeance of about 125 Darcy /mm.

Sample BS-9: A further apertured formed film with straight (i.e. non-slanted) cones, as available from Tredegar under the designation 515FP, gave upon testing according to the “Textiluhr nach Kretschmar” test a permeance of about 275 Darcy /mm.

Similarly, the topsheet material must have a sufficient permeability, and should not impede liquid passage to the absorbent structure.

As can be seen from the above results for backsheets, non-woven materials generally exhibit high gas permeance values, and thus conventional materials, such as described in EP-A-0.774.242 (Palumbo), which is incorporated herein by reference, do not exhibit a major resistance to gas flow

Particularly preferred tospheet materials for applications whereby more or less solid excretions can be deposited on the article, are nonwovens comprising apertures, at least in the portions thereof, which are aligned with the feces deposition region of the article, such as described in more detail in EP-A-0.714.272 or EP-A-0.702.543, and both of which are incorporated herein by reference. Optionally, and preferably for feces handling articles, such topsheets can be combined with feces handling members e.g. underlying such topsheets, and further described in these applications.

The further elements of the article should not limit the convective transport of the discussed elements, but—as far as these are in the convective flow path—be at least as open as the flow limiting elements. This is particularly relevant for means to enhance the integrity of the structure, such as adhesive or other bonding means, or fixation means such as tapes and/or landing zone materials, which may be attached to the outside of the article. This is also relevant for liquid barriers, such as the leg cuffs or so called barrier cuffs. In a particular aspect, when such cuffs are longitudinally sealed liquid impermeably to the topsheet, and preferably therethrough, the underlying core structure should exhibit the describe permeance requirements at least across the width of the article between these tack-down seals of the cuffs.

Beyond not limiting convective flow, further elements can be included, which increase the convective flow. For example, bellows can be incorporated into the article, such as described in the co-filed patent application “ABSORBENT ARTICLE HAVING A BELLOWS FOR CIRCULATING FRESH AIR” (Seitz/Krebs), which is incorporated herein by reference.

In addition to high permeance values, and to particular basis capacity requirements, preferred articles according to the present invention should be comfortably thin and soft, and thus should have a caliper of less than 9 mm at their thickest portion, and a bulk softness value of less than 10 N, preferably less than 5 N and even more preferably of less than 3 N, when tested according to the test method as disclosed in PCT application, filed on Mar. 10, 2000, attorney docket CM2295, titled “Absorbent articles exhibiting improved buckling and bending softness”, which is incorporated herein by reference

### Methods and Determination

General Conditions and Synthetic Urine

Unless otherwise noted, all tests are carried out at about 22 +/−2° C. and at 35 +/−15% relative humidity. The synthetic urine used in the test methods is 0.9% solution of NaCl in distilled water.

Caliper

The caliper of the sample (dry or loaded) is measured (if necessary after a equilibration period) under the desired compression pressure for which the experiment will be run by using a conventional caliper gauge (such as supplied by AMES, Waltham, Mass., U.S.) having a pressure foot diameter of 1⅛″ (about 2.86 cm), exerting a pressure of 0.2 psi (about 1.4 kPa) on the sample, unless otherwise desired and notified.

PMI Gas Permeability

A suitable permeability method for highly permeable materials or structures, especially for materials having a certain caliper of thickness, uses a Capillary Flow Porometer as supplied by Porous Materials Inc., Ithaca, N.Y., U.S. under the designation CF -120 AEXI, with appropriate manuals and software (Version 6.0, CapWin Version 6.54.25; CapRep Version 6.56.15; CapGraph Version 1.5.1) or equivalent.

When following the operation instructions for determining gas permeability as outlined in the user manual, the particular settings have been utilized:

The selected gas is air. The active sample diameter is set to 45 mm. The cylindrical sample can be dry or can be wetted. A spacing insert (of 270.82 g) is be applied without further compressing the sample. The resulting permeability will be expressed in Darcy.

### Kretschmar Textiluhr

The air permeability is determined by measuring the time in which a standard volume of air is drawn through the test specimen at a constant pressure and temperature. This test is particularly suited to materials having relatively high permeability to gases, such as nonwovens, apertured films and the like.

The test is operated in a temperature and humidity controlled environment, at 22±2° C. and 35%±15% relative humidity. The test specimen has to be conditioned for at least 2 hrs.

The test equipment as manufactured by Hoppe & Schneider GmbH, Heidelberg, Germany, under the designation “Textiluhr nach Kretschmar”, is essentially a bellows in a vertical arrangement, with its upper end being mounted in a fixed position, and the lower end being releasably hold at its upper position, which can be loosened by means of a release handle to slide under controlled conditions to the lower position, thereby increasing the volume inside the bellows by pulling air through the test specimen which is covering the air entering opening at the upper end of the bellows. The test specimen is firmly held to cover the air entering opening by means of a fastening ring of 5 cm² or 10 cm² to allow for different samples sizes and/or different permeability ranges. If the 10 cm² ring is used, the sample should be at least 55 mm wide, for the 5 cm² ring at least 35 mm. For both, the samples should have a length of about 150 mm.

In case of very high permeability materials, the opening can be further reduced, with appropriate adjustments to the equipment and calculation.

The equipment comprises a stopwatch (1/100sec) which automatically measures the time between the operation of the release handle thus starting the sliding of the bellows, and the bottom of the bellows reaching its lower end position.

The air permeability k of the material can then be calculated as follows:

k=(V*μ*d)/(t*A*Δp)

wherein

V is the volume of the bladder, here 1900 cm³;

μ is the viscosity of the air, here 1.86*10⁻⁵ Pa see;

d is the test specimen caliper in mm;

t is time required for the expansion of the bellows, in sec;

A is the air entering opening, here 4.155 cm²;

Δp is the pressure differential, here 160 Pa.

The resulting unit of k is cm² , whereby 1 Darcy corresponds to 9.869*10⁻⁹ cm².

The test is repeated once for each test specimen, and should be repeated on 10 specimen to provide a representative basis for a material.

As discussed in the above, the present invention aims at providing permeable materials without necessitating the need for creating particular convention channels. Consequently, the above mentioned permeability test (and the respective permeance measurement as described below) should aim at determining the permeability of these structures, and henceforth, the above tests may need certain modifications so as to measure the storage material rather than the apertures, such as by reducing the test specimen opening, or—if readily achievable—by blocking some of the apertures.

Permeance

Permeance is defined as the permeability (as determined in the above) per unit thickness of the material, expressed in Darcy/mm.

Polyhole Test

One piece of 10 cm by 10 cm of Filterpaper such as Grade Medium White W/S available from Schleicher & Schüll, Germany is weighed to the nearest 0.001 g.

On a suitable flat surface, such as a lab bench, an absorbent article is placed flat over the filterpaper, such that the loading point on the topsheet of the article faces upwards, and the filterpaper is centered under this loading point, in direct contact with the backsheet of the article, or the material to be tested.

The sample is loaded at the loading point for its intended use with an appropriate volume of liquid, preferably 0.9% by weight saline solution, generally about 80% of its theoretical capacity. If this is not determined, following values can be used, exemplifying the loading for various, broadly used baby diaper sizes:

| | | |
|---|---|---|
| Mini/Mini plus | (size 1, 2) | 175 ml |
| Midi | (size 3 | 250 ml |
| Maxi | (size 4) | 300 ml |
| Maxi plus/larger | (size 5, 6) | 350 ml |

The loading of the article is executed by pouring it through a funnel, whereby the outlet is positioned 20 mm above the loading point of the absorbent article. A suitable funnel for baby diaper applications has funnel diameter of about 82 mm (about 3.1 inch), a funnel height of about 132 mm (about 3.5 inch), and an outlet tube of about 70 mm (about 2.7 inch) length, and about 6.7 mm (about 0.25 inch) inner diameter. The flow rate of liquid into the funnel should be fast, but it should be adjusted by controlled pouring of liquid so as to avoid excessive pooling or run-off outside of the article during the loading.

After addition of the liquid, and a further a waiting period of 60 secs (+/−3 sees), a rectangular weight (10 cm*10 cm; each +/−3 mm) of 3.65 kg +/−0.5%. After 120 sec (+/−3 sees), as can be measured by any suitable timer, the weight is removed and the filterpaper is re-weighed to determine the liquid-pick up.

The weight pick up is reported to the nearest 1 mg, and then converted into and expressed as fluid absorption in ml.

Dynamic Liquid Impact Test

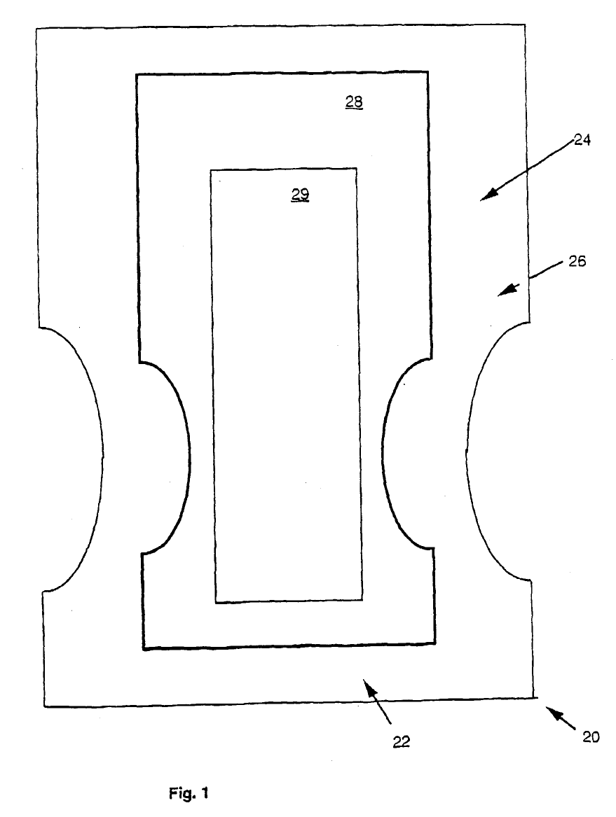

Dynamic fluid transmission is measured with the apparatus 9100 shown in FIG. 1. According to this test, an absorption material 9102 weighed to the nearest 0.0001 gram is placed directly on top of the energy absorbing impact pad 9103. The absorption material 9102 may comprise a No. 2 filter paper available from Whatman Laboratory Division, Distributed by VWR Scientific of Cleveland, Ohio. The absorption material should be able to absorb and retain simulated urine which passes through the sheet material being tested. The energy absorbing impact pad 9103 is a carbon black filled cross linked rubber foam. The 12.7 cm by 12.7 cm (5 inch by 5 inch) square impact pad has a density of 0.1132 g/cm³ and a thickness of 0.79 cm (0.3125 inches). The impact pad 9103 has a Durometer Value of A/30/15 according to ASTM 2240-91. A circular absorbent core material 9104 measuring 0.0635 meters (2.5 inches) in diameter is weighed. The absorbent core material may comprise individualized, crosslinked wood pulp cellulosic fibers as described in U.S. Pat. No. 5,137,537 issued to Herron et al. on Aug. 11, 1992.

The absorbent core material should be able to hold a sufficient amount of simulated urine, e.g., at least about ten times its dry weight.

Other absorbent materials that can be used include airfelt, tissue, cellulose wadding, as long as these exhibit the required absorbent capacity of at least 10 g/g. If the materials have a capacity below 10 g/g then they should be wetted to at least 80% of their saturation capacity. Also, the absorbent materials should be essentially free of “superabsorbent materials” which might bind the liquid too tightly and thus affect the results.

The absorbent core has a basis weight of about 228 g/m². The absorbent core material is then is loaded with simulated urine to about ten (10) times its dry weight. The simulated urine is an aqueous 0.9 % by weight saline solution, exhibiting a surface energy value as conventionally determined of 72.5 mN/m.

A section of the backsheet material 9105 to be tested is placed face down with the outside surface on a clean and dry tabletop. The loaded core material 9104 is placed directly in the center of the backsheet material 9105 . The backsheet/core arrangement is then secured to the impact portion 9107 of the impact arm 9108 with a rubber band 9109. The backsheet/core arrangement is positioned such that the core 9104 is adjacent the bottom surface 9110 of the impact portion 9107. The impact arm 9108 is raised to a desired impact angle to provide the desired impact energy. The impact arm 9108 is dropped and the impact arm 9108 is then allowed to rest on the sample for about two minutes after impact. The arm is then raised and the filter paper 9102 is removed and placed on a digital scale. The mass of the wet filter paper is then recorded at the three minute mark. The dynamic fluid transmission value (DFTV) is calculated and expressed in g/cm² using the following formula:

DFTV={mass of the wet filter paper (grams)−mass of the dry filter paper (grams)}/{impact area (m²)}

The impact area, expressed in m², is the area of the bottom surface 9110 of the impact portion 9107. The impact area is 0.00317 m . The absorbent core material 9104 should have an area slightly larger than that of the impact area of the surface 9110.

Water Vapor Transmission Rate

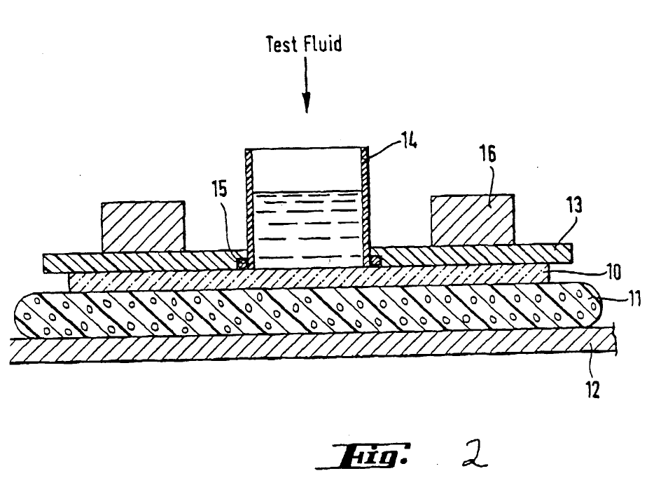

When referring to FIG. 2, the test specimen (210) having a diameter of about 120 mm is positioned centered in a flat out condition over a 75 mm deep cylindrical cup (220) with a circular opening (222) of 50 mm inner diameter, which has been filled up to about 10 mm below the upper end with distilled water (226). The sample is supported by a cylindrical rim (224) at the top of the cup, of about 120 mm diameter. The sample is covered by a cover lid (230) of a inner diameter to fit the outer diameter of the rim. The lid has a centered opening (232) corresponding to the opening 222 of the cup opening, and a flange (240) to allow fixation of sample and to minimize evaporation losses at the side, extending approximately 70 mm. The lid has a weight of approximately 238.5 g.

The assembly is weighed and positioned into a climate chamber, such as available from WTB Binder, Tuttlingen, Germany, type 377200990031.00 at 33° C. 20% RH, with high air circulation rate of about 15 cm/sec air velocity.

After 5 hrs, the assembly is removed from the chamber, and reweighed. The Water Vapor Transmission Rate is calculated from the loss per time unit and opening area (the latter being 1963.5 mm²), and expressed in units of g/m²/24 hrs.

For very different rates, the evaporation time in the chamber can be adjusted, such as to 2 hrs for very permeable materials, or to 24 hrs for materials with low permeability.

WVTR of Full Product

The equipment described above can also be used to determine WVTR of samples having higher caliper such as dry or wet diapers. In this case, a circular sample having a diameter of 109 mm is applied at the cup rim surface. To avoid exchange with the environment, a glass ring having an outer diameter of 120 mm, an inner diameter of 110 mm, and a height of sample caliper minus 1 mm is put around the sample.

When evaluating dry articles, it has to be taken into account that a dry article acts as a desiccant, that is, absorbs water vapor until reaching saturation. This effect can be minimized by equilibrating the sample prior to determining WVTR.

For equilibration, a circular 109 mm diameter cut out piece of the article is placed in a suitable box, backsheet facing the environment. This equipment is placed for about 48 hrs inside a climate chamber of the type as described in the above, at 33° C., 90% RH, maximum ventilation (15 cms). When removing the sample from the chamber, the starting weight of equilibrated article is recorded. As described in the above, the equilibrated piece is then placed on the surface of the cup filled with water with backsheet facing down to the water, topsheet facing to the environment. The glass ring is placed around the sample. The equipment is placed in the climate chamber as above, and after removing it therefrom, the weight of complete equipment is recorded, as well as the end weight of the test specimen, to account for further absorption of vapor or evaporation from equilibrated article through the topsheet to environment.

When evaluating wet articles, it has to be taken into account that wet articles can show significant additional evaporation from loaded core through topsheet to environment. Thus, weight loss of equipment is not only due to diffusion of water vapor from cup through the product to the environment, but also due to evaporation from loaded article through topsheet to environment. Instead of the equilibration as for the dry article, the cut-out piece is now evenly loaded with 10 g Saline per g of the test specimen. The starting weight of the test specimen is recorded accordingly, and so is the starting weight of the filled cup with water only (i.e. no sample).

The loaded test specimen is placed on the surface of the filled cup filled, the backsheet facing down to the water, topsheet facing to the environment, and the protective ring is added to surround the sample.

The equipment is placed in the climate chamber as above, and after removing it therefrom, the weight of complete equipment is recorded, as well as the end weight of the test specimen, to account for further absorption of vapor or evaporation from equilibrated article through the topsheet to environment.

There are two equivalent possibilities of how to calculate WVTR from the above measurements for wet diapers:

=(start weight of cup with water only−end weight of cup with water only)/(Time×opening area);

=((start weight of complete equipment−end weight of complete equipment)−(start weight of wet diaper−end weight of wet diaper))/(Time×opening area).

Wet Article Relative Humidity Differential

Temperature and relative humidity vary between body sites under the diaper due to loading pattern, babies activity and emotional state, and environment, such as room conditions. A multi point measurement provides the opportunity to monitor simultaneously conditions at several locations underneath the diaper.

Particular interest lies in the understanding of change in conditions between locations corresponding to the loaded and non-loaded areas of the diaper. Typical diaper users change the diaper between 3 to 12 hours. Within this period on average the baby has loaded the diaper with 3-4 gushes of urine. Therefore, a partially loaded diaper may be worn for several hours before being changed. Thus, the conditions under the article when worn, i.e. in the space between the article and the skin of the wearer, are monitored at predetermined locations of the sensors in this space.

The microclimate as a function of body temperature and water evaporation may also change in response to babies activities and emotional state. To correlate potential microclimate changes babies can be supervised during the measurement period by their parent(s)/guardian(s) who record specific events, activities and times in a diary.

Further, the micro-climate within the article is dependent on the room conditions. Henceforth, it has been found particularly useful to include a reference measurement point on the wearer, but not covered by the article which is evaluated, but only by normal clothing, e.g. conventional underwear.

The present method and the particular equipment used herein should be set up with particular consideration of safety and hygienic conditions, such as the Declaration of Helsinki Recommendations guiding physicians in biomedical research involving human subjects, as adopted by the 18th World Medical Assembly, Helsinki, Finland, June 1964 and its further amendments.

The Temperature and relative Humidity sensoring device consists of temperature (T) and relative humidity (RH) sensors, data-loggers to store data and a harness carrying the sensors and the data-logger.

Fitting the Temperature and Relative Humidity Monitoring System as described in more detail hereinafter, and changing diapers will preferably take place in a separate room. Before fitting the Monitoring System to the baby the sensors and cables will be fixed onto the harness with medical tape. The cables will be connected to the data-logger. Excess cables and the data loggers will be stored and fixed in the data-logger bag at the back of the harness.

The complete harness will be fitted on the baby with the help of the parent and/or guardian during the change of the diaper. The Monitoring System is fitted onto the baby such that all sensors on the harness face the diaper side. The elastics of the harness will be carefully adjusted to the baby to avoid skin marks. Following the adjustment of the harness a diaper is fitted over the Temperature and Relative Humidity Monitoring System carefully avoiding dislocation of the sensors. After fitting and diaper change the baby will be taken to a separate room for the wear period. The babies may not wear underwear over the diaper, if climatic conditions allow.

Monitoring System Wearing Period

The Monitoring System wearing period may last up to 12 hours. During the wearing period of the Monitoring System and the diaper the babies can be entertained by their parent and/or guardian. Babies may be encouraged by parents or guardians perform playful tasks (i.e. like in play groups) and/or to act as they like. A diary of the activities can be kept by the parent and/or guardian as appropriate for the specific study objective. Alternatively, the activities can be recorded on video for evaluation of activities at a later time.

Controlled Diaper Loading

Depending on the specific study objective diapers may be loaded with cumulative gushes of “artificial urine” (i.e. physiological saline) up to the desired representative loading in view of the design and intended use of the article. For example, to represent an overnight usage of a MAXI size diaper (i.e. intended for an about 9 to 18 kg baby) a total volume of 300 ml has been found suitable. The “artificial urine” will be prepared as described herein and warmed up to 37° C. prior to loading. Diapers may be pre-loaded immediately to fitting or loaded in-use. Loading in-use will be performed using soft flexible tube with rounded tip at a controlled loading rate and volume.

Study Size

It is estimated that in total approximately 5 babies will need to be recruited to provide a meaningful basis. Selection criteria may be set, such as relating to generally healthy babies of both sexes, weighing more than 7 kg and being elder than 6 months (corresponds to Maxi- or larger size users).

Temperature & Relative Humidity Monitoring System Information

The monitoring system comprises three essential elements, namely a harness for fixing the system on the wearer, the sensors for measuring temperature and relative humidity, and the data logging system.

The harness is designed to allow accurate positioning and fixing of the sensors on the wearer, and to provide means for carrying the data logging system. The harness needs to be made of skin friendly material. Materials described below are combined as in the following configuration.

A suitable design for the harness has been found by comprising a waist belt to be fitted around the waist of the wearer, and further comprising fixation elements fitted between the legs of the wearer.

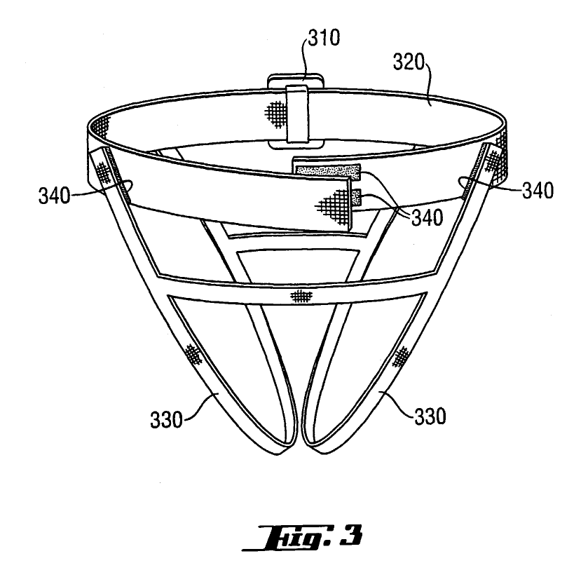

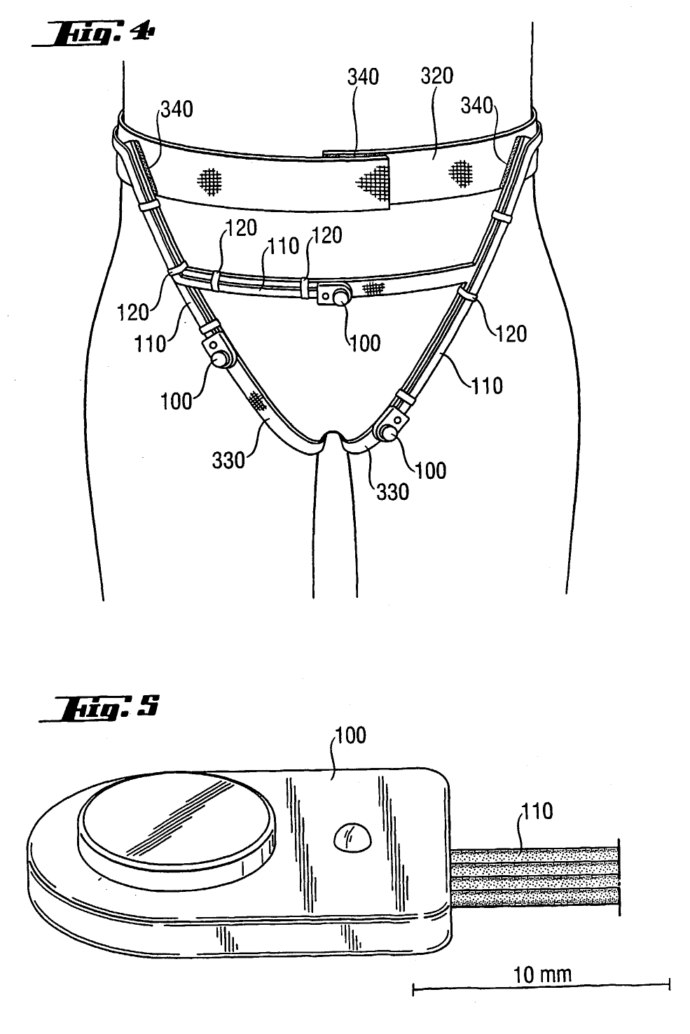

A typical harness can be seen in FIG. 3 as a schematic diagram, and in FIG. 4 as a photograph fitted on a baby mannequin.

Sensorboxes and cables are fixed on the harness with an adhesive tape, such as LEUKOSILK™. The bag for the data loggers is made out of cotton. It is equipped with 4 snap-fasteners to be able to remove the data loggers. The bag is tightened to the harness via mechanical fasteners. Data loggers are additionally coated by PU foam to achieve more comfort.

Further particularly suitable materials have been found as useful as follows. The skilled person will be readily enabled to replace materials by equivalent ones, or to adjust sizes to other sizes of the wearer, except for the sensors location, which is an essential element of the present invention.

An athletic supporter (“jock-strap” or “Tiefschutz”) (320) such as available from Adidas, AG, Germany, is sewn together with Rubber straps (330) such as available from Wenco Service Marketing, Duesseldorf, Germany, as “Baby Elastic” and conventional “popper buttons” to the described harness. Medical tapes, such as available form Beiersdorf AG, Hamburg, Germany, and Velcro™ type hook and loop closure systems (340) are used to close the harness and to fix it at the wearers body. Conventional cotton fabric can be used as bag for the data logger (310).

The harness can be replaced by other means to fix the sensors, and the logger appropriately, such as by stretch-pants, or topical adhesives applied to fix the sensors directly on the skin of the wearer, as long as this fixation means does not impede the functionality of the article, and it should further have a minimal effect on the climate within the article, and on the skin.

Data Acquisition

The climate data as generated by the sensors as described herein are gathered by a data logging system worn by the wearer, or a data transmission system connected to a data logging system physically located away from the wearer. The connection between the transmission system and the data logging system is preferably not executed by fixed cables, but rather by cable-less systems, such as radio signals or infra-red data transmission systems.

A particularly suited system includes a data-logging system to be worn by the tested person, wherein the data are recorded during the test period, and from where these data can be read into a data processing unit after the testing.

A specific example is Smart Reader Plus, available from Status Instruments Ltd, Tewkesbury, United Kingdom, connected via an insulated flat four wire cable such I.D.C. Flachbandkabel, form RS Components GmbH Mörfelden-Walldorf, Germany.

Sensors and Sensor Box

The sensors are particularly designed to measure both temperature and relative humidity at small dimensions, and compatible with hygienic and safety requirements during the testing.

The sensors used for the Monitoring System should have the following accuracy, which should be maintained during data logging, transmission, and/or processing.

| Temperature: | ±0.2° C. |
|---|---|
| Relative Humidity: | ±2% |

A suitable temperature sensor is a precision thermistor, as from Omega Precision Thermistor Resistance Omega Engineering Inc., Stanford, USA.

As suitable relative humidity sensor is Capacitive Humidity Sensor, such as available from OHMIC Instruments Co., Maryland, USA, under the designation HC 700.

The sensors can be affixed to the harness by conventional, such as dispersion adhesives, shrinking tubes, and/or casting polymers, such as of the ABS type.

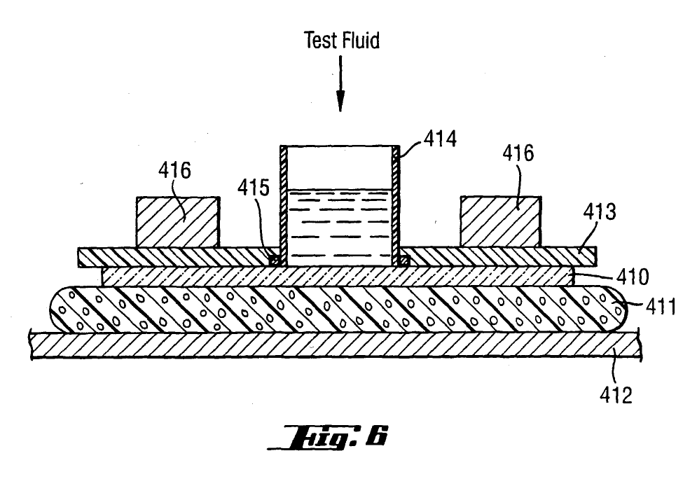

All relative humidity sensors are cast into a plastic box (17×11×4 mm, see FIG. 5, hereafter called sensor box). Six out of seven temperature sensors are also included in these sensor boxes. The remaining temperature sensor is isolated with a shrinking tube and will be placed inside the absorbent part of the diaper. The sensor boxes are connected with the data loggers via a 4-wire isolated cable.

Cleaning Agents

Before usage, all sensor boxes will be disinfected by 15 minutes incubation in a 6% solution of Gigasept FF in distilled water. Afterwards, they will be washed by immersion for 5 minutes in distilled water and reused when they are dry again. If there is any contamination of baby's excrement, sensors will either be cleaned before disinfecting or discarded.

The harness can washed in a regular washing machine with conventional washing powder at a temperature of 95° C.

Electrical Parts

Two coupled data loggers are stored in the data logger bag at the rear side of the harness. Data loggers do not come in direct contact with the skin. For improved wearing comfort the data loggers are wrapped in a soft PU foam material.

Each data logger unit is powered by one 3 V battery of about 24 mm diameter. The batteries are secured inside the data loggers with a firm clip. Furthermore, the four point clip button closure system of the data logger bags provides additional protection from accidental access to batteries. Constant supervision of parents or guardians will further ensure that babies have no access to the batteries.

The cables connecting the data loggers with the sensor boxes are as small in width as the harness elastics and are fixed via the medical tape to that side of the elastic which is not in contact with the skin. The cable insulation consists of flexible PVC. PVC is an inert polymer with a good safety profile and used in commercial and medical applications (e.g. urine bags, flexible catheters).

The sensor boxes contain the temperature and relative humidity sensor. The relative humidity sensor lies protected under a cover made of a coated nickel alloy. The sensor boxes are fixed to the harness on that side which is not in contact with the skin.

The following describes a typical set up for carrying out the test, though of course particular elements such as of the babies activities, clothing etc. can be varied. Preferably, babies should be healthy, and the number of babies in one group should be kept small, such as 5 babies. Normal hygienic precautions should be taken, such as washing equipment and clothing used on the babies and using hand disinfectants. Equipment vulnerable to damage in machine washing (e.g. electrical parts such as sensor boxes and wires) will be disinfected by immersion in a solution of a disinfectant.

Positioning of the Sensors

The Temperature and Relative Humidity monitoring system consists of Harness with temperature (T-) and relative humidity (RH-) sensorboxes and/or pure RH sensor, to be worn underneath the article. Preferably, five T-/RH sensors and one RH sensors are used. A further T-sensor is further placed inside the article. A further reference sensor is applied outside the article.

The T-sensor is applied from the outside after the article is applied on the wearer and the sensor is applied through the backsheet to be positioned closely in the loading region of the article, affixed e.g. by adhesive tape.

The reference sensor is applied outside the article in the rear waist part at the right hip such as by mechanical fastener. Since the part is not underneath the hygienic article, the reference sensor is located on the harness towards the outside of the harness (i.e. on the opposite side to the one oriented towards the skin of the wearer) and is covered by the cotton clothing.

For the determination of the result of the measurement, the signals of the sensors covered by the absorbent article are averaged, expressed and calculated to a tenth a percent.

To determine the Wet Article Relative Humidity Differential, the relative humidity value of the reference sensor (i.e. the one not covered by the article, but by terry cloth only) is deducted from the average relative humidity values.

Under most circumstance, the result will be a positive value. In case that the coverage by the article will result in a reduced relative humidity, the result will be negative.

Evaporation Rate from Loaded Absorbent Article

This test method relates to an absorbent article. A rectangular test specimen of 70 mm (in transverse direction of the article) by 100 mm (in longitudinal direction of the article) is cut by suitable scissors or a cutting blade from a representative part of the absorbent core, such as transversely centered, and from about 6 cm from front core edge.

The dry weight is recorded, and the specimen is placed in a glass box of about 72 mm by 102 mm, and about 40 mm high without lid, with backsheet down, and the topsheet facing to environment.

The specimen is loaded with 10 g of 0.9% saline solution per gram weight of the specimen, whereby the liquid is evenly distributed over the area, thereby avoiding the wetting of the glass box.

The complete weight of the glass box with the loaded specimen is recorded.

The equipment is placed into a climate chamber such as available from WTB Binder, Tuttlingen, Germany, type 37720099003100 at 33° C.+/−2°, at 50% relative humidity (RH) +/−3%. The ventilation is adjusted to provide an air flow velocity of about 15 cm/sec over the opening of the glass box.

After two hours evaporation time, the end weight of the complete glass box with the specimen is recorded.

The area specific evaporation rate is determined

Evaporation Rate=(Start weight−End weight)/(Time×sample area).

whereby the start and end weight is the total weight of the glass box with the specimen.

The above loading values have been found useful for baby diapers, especially for baby diapers for babies of the size of about 9 to 18 kg, often referred to as MAXI size. In case of very different absorbent capacities of the absorbent article under consideration, the amount of liquid load should be adjusted to about 50% of the theoretical basis capacity as defined herein.

Acquisition Test This test should be carried out at about 22+/−2° C. and at 35+/−15% relative humidity. The synthetic urine used in these test methods is 0.9 % Saline solution.

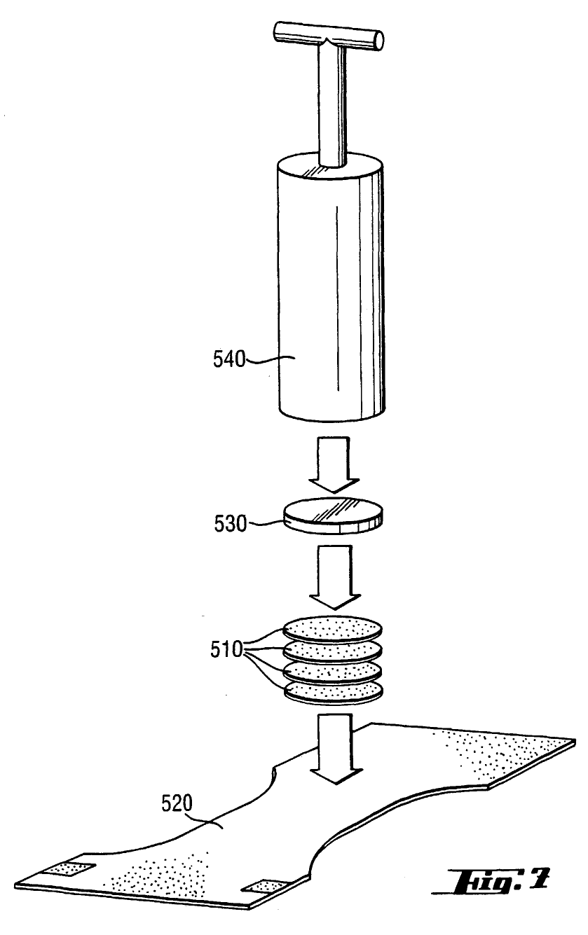

Referring to FIG. 6, an absorbent structure (410) is loaded with a 75 ml gush of synthetic urine at a rate of 15 ml/s using a pump (Model 7520-00, supplied by Cole Parmer Instruments., Chicago, U.S.A.), from a height of 5 cm above the sample surface. The time to absorb the urine is recorded by a timer. The gush is repeated at precisely 5 minute gush intervals until the article is sufficiently loaded. Current test data are generated by loading four times.

The test sample, which can be a complete absorbent article or an absorbent structure comprising an absorbent core, a topsheet, and a backsheet, is arranged to lie flat on a foam platform 411 within a perspex box (only base 412 of which is shown). A perspex plate 413 having a 5 cm diameter opening in its middle is placed on top of the sample on the loading zone of the structure. Synthetic urine is introduced to the sample through a cylinder 414 fitted, and glued into the opening. Electrodes 415 are located on the lowest surface of the plate, in contact with the surface of the absorbent structure 410. The electrodes are connected to the timer. Loads 416 are placed on top of the plate to simulate, for example a baby's weight. A pressure of about 50 g cm-2 (0.7psi) is achieved by positioning weights 416, e.g. for the commonly available MAXI size 20 kg.

As test fluid is introduced into the cylinder it typically builds up on top of the absorbent structure thereby completing an electrical circuit between the electrodes. The test fluid is transported from the pump to the test assembly by means of a tubing of about 8 mm diameter, which is kept filled with test fluid. Thus the fluid starts to leave the tubing essentially at the same time the pump starts operating. At this time, also the timer is started, and the timer is stopped when the absorbent structure has absorbed the gush of urine, and the electrical contact between the electrodes is broken.

The acquisition rate is defined as the gush volume absorbed (ml) per unit time(s). The acquisition rate is calculated for each gush introduced into the sample. Of particular interest in view of the current invention are the first and the last of the four gushes.

This test is primarily designed to evaluate products generally referred to as MAXI size products for a design capacity of about 300 ml, and having a respective Ultimate Storage Capacity of about 300 ml to 400 ml. If products with significantly different capacities should be evaluated (such as can be envisaged for adult incontinence products or for smaller babies), the settings in particular of the fluid volume per gush should be adjusted appropriately to about 20% of the total article design capacity, and the deviation from the standard test protocol should be recorded.

Post Acquisition Collagen Rewet Method (Refer to FIG. 7)

Before executing the test, the collagen film as purchased from NATURIN GmbH, Weinhein, Germany, under the designation of COFFI and at a basis weight of about 28 g/m² is prepared by being cut into sheets of 90 mm diameter e.g. by using a sample cutter device, and by equilibrating the film in the controlled environment of the test room (see above) for at least 12 hours (tweezers are to be used for all handling of the collagen film).

At least 5 minutes, but not more than 6 minutes after the last gush of the above acquisition test is absorbed, the cover plate and weights are removed, and the test sample (520) is carefully placed flat on a lab bench. 4 sheets of the precut and equilibrated collagen material (510) are weighed with at least one milligram accuracy, and then positioned centered onto the loading point of the article, and covered by perspex plate (530) of 90 mm diameter, and about 20 mm thickness. A weight (540) of 15 kg is carefully added (also centered). After 30+/−2 seconds the weight and perspex plate are carefully removed again, and the collagen films are reweighed.

The Post Acquisition Collagen Rewet Method result is the moisture pick up of the collagen film, expressed in mg.

It should be noted further, that this testing protocol can be adjusted easily according to specific product types, such as different baby diaper sizes, or adult incontinence articles, or catamenial articles, or by the variation in the type and amount of loading fluid, the amount and size of the absorbent material, or by variations in the applicable pressure. Having once defined these relevant parameters, such modifications will be obvious to one skilled in the art. When considering the results from the adjusted test protocol the products can easily be optimizing these identified relevant parameter such as in a designed experiment according to standard statistical methods with realistic in use boundary conditions.

Drip Capacity

The drip capacity test described here is based on a standard and industry wide applied raw material test for airfelt (fluff) pulp. The test was initially developed to evaluate the degree to which a fibers can acquire, transport (distribute) away from the loading point and retain a load of synthetic urine in a fiber web. A slight modification of the test is used to simulate more in-use conditions.

In the acquisition-drip test a 75 ml gush of synthetic urine (0.9% saline) is applied to a fiber web supported on a wire mesh (porous) at a rate 15 ml/sec. The (saturated) drip capacity is then determined from the fluid that is retained in the fibrous material after the gush.

To execute the test, a sample pad 7.5 cm×25 cm is weighed and placed on a large mesh wire screen positioned on a drip tray (like in the diagram) which is then mounted on a weight balance. 75 ml of Synthetic urine is introduced via a pump (the same pump used and detailed in the acquisition test) into the center of the sample at a rate of 15±0.25 ml/sec.

By suspending the mesh screen on a balance one can determine closely the amount of urine retained by the sample and urine passed into the drip tray. This helps to minimize variations of the pump delivering the urine.

Note the pump delivery rate is confirmed prior to each run.

The drip capacity is then given as the ratio:

Urine retained on saturation (ml)

Dry Weight of sample (g)

Optionally, the “drip time” can be recorded, i.e. the time difference between the start of loading the structure and the time when the first drop falls out of the sample.

What is claimed is:

1. Absorbent article, comprising an absorbent core, a topsheet, and a backsheet, wherein the absorbent core and the backsheet have a convective air permeability, and wherein the article has Wet Article Relative Humidity Differential (WA-RH-D) of less than 20%,

2. The absorbent article according to claim 1 wherein the article has Wet Article Relative Humidity Differential (WA-RH-D) of less than 15%.

3. The absorbent article according to claim 1 wherein the article has Wet Article Relative Humidity Differential (WA-RH-D) of less than 10%.

4. Absorbent article, comprising an absorbent core, a topsheet, and a backsheet, wherein the absorbent core provides a basis capacity of at least 0.7 ml/cm² and the backsheet provides a Dynamic liquid impact performance of less than 20 g/m².

5. Absorbent article according to claim 4, wherein the backsheet provides a polyhole rewet of less than 10 mg.

6. Absorbent article according to claim 4, wherein the absorbent core has a ultimate storage capacity of less than 1.5 times the design capacity.

7. Absorbent article comprising a topsheet, a backsheet, and an absorbent core, the absorbent core comprising at least a core region having an average basis capacity of at least 0.3 ml/cm², the core region having a dry permeance of at least 1.0 Darcy/mm, and a wet permeance of at least 0.1 Darcy/mm; wherein the article has a wet permeance of at least 0.1 Darcy/mm.

8. Absorbent article according to claim 7, wherein the core region has a wet permeance of at least 3 Darcy/mm.

9. Absorbent article comprising a topsheet, a backsheet, and an absorbent core interposed between the topsheet and the backsheet, the absorbent core comprising a liquid storage region and a liquid acquisition/distribution region positioned between the liquid storage region and the topsheet, wherein the acquisition/distribution region comprises an evaporation barrier layer/region, and wherein the absorbent article provides an evaporation value of less than 30% when submitted to an Evaporation test.

10. Absorbent article according to claim 9, wherein the acquisition/distribution region has a drip capacity of at least 5 g/g.

11. Absorbent article according to claim 9, wherein the acquisition/distribution region comprises cellulosic material.

12. Absorbent article according to claim 9, further comprising a bellows, which is repeatably deformable to force air flow through the absorbent article in a controlled manner.

13. Absorbent article according to claim 9, wherein the article provides a Polyhole rewet of less than 0.10 mg.

14. Absorbent article according to claim 9, wherein the absorbent article is fitted on a wearer, thereby defining a space between the topsheet and the wearer, the space exhibiting an micro climate having a Relative Humidity of less than 50% RH.
